(19) United States
(12) Patent Application Publication
(10) Pub. No.: US 2017/0020091 A1
(43) Pub. Date: Jan. 26, 2017

(54) Method and System for Automated Data Analysis of Soil Moisture

(71) Applicant: AquaSpy, Inc., San Diego, CA (US)

(72) Inventor: Thomas McCoid, San Diego, CA (US)

(73) Assignee: AquaSpy, Inc., San Diego, CA (US)

(21) Appl. No.: 15/214,332

(22) Filed: Jul. 19, 2016

Related U.S. Application Data
(60) Provisional application No. 62/194,766, filed on Jul. 20, 2015.

(51) Int. Cl. A01G 25/16; G01N 33/24; G01N 27/12

(52) U.S. Cl. CPC A01G 25/167; G01N 27/121; G01N 33/246

(57) ABSTRACT

A Method and System for Automated Data Analysis. The system and method provides advanced and accurate soil moisture information along the depth of soil moisture probes. The system and method provides the farmer with up to date root depth data that is derived from being able to recognize that there is water being consumed at a particular depth. Once root depth is derived, the system is able to determine the fill point for the soil and crop. Refill point is also be able to be provided by the systems advanced detector sensitivity and analytical capability.

This application is filed within one year of, and claims priority to Provisional Application Ser. No. 62/194,766, filed Jul. 20, 2015.

### BACKGROUND OF THE INVENTION

1. Field of the Invention

This invention relates generally to agricultural monitoring apparatus and, more specifically, to a Method and System for Automated Data Analysis of Soil Moisture.

2. Description of Related Art

As global population continues to grow, the need for an analytical, scientific approach to crop growth has become of increasing importance. While many environmental conditions are variable, and only controllable in the context of choice of locale for a particular planting, some can be controlled.

One environmental condition affecting crop growth and yield that has long been sought to be “controlled” is that of irrigation. All farmers know that a key to a good crop is to irrigate enough to promote good growth, but not so much as to overwater the plants or to dilute fertilizers and/or soil nutrients.

For the small “family” farm, optimizing irrigation, while inexact, is achievable through persistence and long-term direct experience. This situation does not typically apply to the large “corporate” farm, however. The corporate farm is much larger than the traditional family farm, and consists of enormous planted areas that can only be planted, tended and harvested by mechanized means.

The irrigation of these corporate fields is handled by automated systems that dispense water (at times mixed with fertilizer and other compounds) over the planted areas, usually by extremely long irrigation arms pivoting around the field. These irrigation arms now have the ability to regulate the amount of water dispensed in a very controlled fashion along the length of the arm, as well as for particular arc sectors while the arm pivots around the field.

The key to the efficient operation of these sophisticated irrigation arms is the ability to detect and report the actual moisture levels in the soil being irrigated. This data can then be fed to the irrigation system for the purpose of responsively adjusting the amount of irrigation water dispensed over a particular portion of the planted field. Soil moisture sensors have continued to evolve for just this purpose. FIG. 1 depicts a conventional version of just such a device.

FIG. 1 is a cutaway side view of a conventional soil moisture probe 10¹. The probe 10 is usually an elongate, cylindrical device that is buried beneath the soil 8 at strategic locations around the monitored area of a field. A communications conduit 16 (generally a multi-filament cable having a waterproof cover, however wireless conduits are also in development) interconnects the probe 10 and a central control unit (not shown). The control unit directs the operation of the network of probes 10, records their soil moisture data (and usually other parameters), and transmits the data wirelessly to the group operating the probes 10. ¹ As used throughout this disclosure, element numbers enclosed in square brackets [ ] indicates that the referenced element is not shown in the instant drawing figure, but rather is displayed elsewhere in another drawing figure.

The probe 10 has a cylindrical housing 14, typically made from a section of PVC pipe (or other suitable material). The typical housing 14 has a wall thickness of approximately 2.4 mm, to insure sufficient structural integrity to protect the internal electronics against crushing or intrusion from other elements in its environment. The ends of the housing 14 are capped and sealed.

A string of moisture sensors 12 are encapsulated within the sealed housing 14, and in electrical communication with the wiring in the communications conduit 16. Each sensor 12 creates an individual EMF field perpendicular to the housing 14 (in the surrounding soil 8). These fields are capable of reading the electrical conductivity of the surrounding soil. Each sensor 12 is in regular, known, spaced relation along the length of the housing 14. By burying the probe 10 aligned vertically at a known depth, it is possible, then, to measure the conductivity of the soil 8 at selected depths (e.g. D1, D2, D3, etc.) in close proximity to the probe 10. Basic representations of these conductivity curves 50 are shown in FIG. 2. As will be discussed further below, each sensor [12] has an individual curve (e.g. 50(1), 50(2), etc.) that plots conductivity (moisture content) to time. These curves provide the farmer with a historical record of the moisture content of their field at varying standard depths.

Turning to FIG. 3, we can examine a useful application for this soil moisture data. FIG. 3 is an example of a typical irrigation template 20. Irrigation templates are specific to the typical soil, environmental conditions, and crop type for a particular locale. Here, we have a template 20 for corn—it plots the typical water use of the corn plant as it goes through its growth cycle. This information is valuable because the farmer can assume (at least generally) that if the soil [8] in the field is maintained with the same moisture as is required by the plant at its particular growth stage, it will promote optimum growth in the crop.

The corn growth process can be broken up into five plant stages: (1) first planted; (2) V12 (a measure of the number of leaves on the stalk); (3) the tassel stage; (4) dent formation; and (5) maturity. These stages are simply provided as an example—other growth stages have also been identified.

Through extensive data recording for a particular geography, it is possible to predict how much water that the corn plants in a field will consume as they grow. This is plotted here as the irrigation curve 22. Because there is some variability in a particular soil and plant instance, the typical template 20 also defines a maximum recommended over-irrigation curve 24. This curve 24 is considered to be the maximum that the plants should be watered without negatively impacting the crops' growth performance. The shaded area between the maximum irrigation curve 24 and the optimum irrigation curve 22 is the optimum irrigation region 26. The goal for any soil moisture and irrigation measurement system is to provide the farmer with the data and advice to allow the farmer to keep the soil moisture level within the optimum irrigation region 26.

While this goal is theoretically possible, real-world implementation has never progressed beyond educated guessing. The problem is that as the plants develop, their roots burrow deeper and deeper into the soil. Irrigation, on the other hand, is typically applied to the surface of the soil. The normal heating and cooling between daytime and nighttime, as well as the effect of sun and humidity injects so much variability into the prediction of soil moisture content at the actual root depth of the plants (which is continually changing), that there has been no prior reliable solution.

What is needed is a reliable system and method that can measure not only the moisture content in the soil at pre-set depths, but that can also predict how much the farmer should irrigate the field in order to provide optimum soil moisture content at that specific root depth.

### SUMMARY OF THE INVENTION

In light of the aforementioned problems associated with the prior systems and methods, it is an object of the present invention to provide a Method and System for Automated Data Analysis of Soil Moisture. The system and method should provide advanced and accurate soil moisture information along the depth of soil moisture probes. This system and method should provide the farmer with up to date root depth data that is derived from being able to recognize that there is water being consumed at a particular depth. Once root depth is derived, the system should be able to determine the fill point for the soil and crop. Refill point should also be able to be provided by the systems advanced detector sensitivity and analytical capability.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings, of which:

FIG. 1 is a cutaway side view of a typical soil moisture probe;

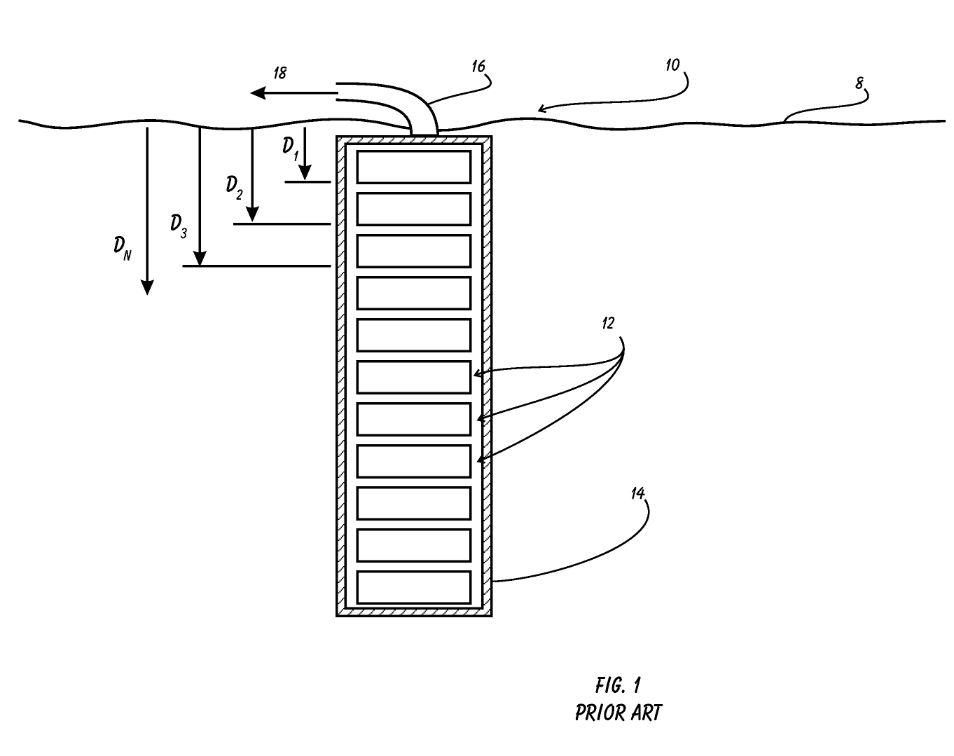

FIG. 2 is an example of a partial set of conductivity (soil moisture) curves for a soil moisture probe;

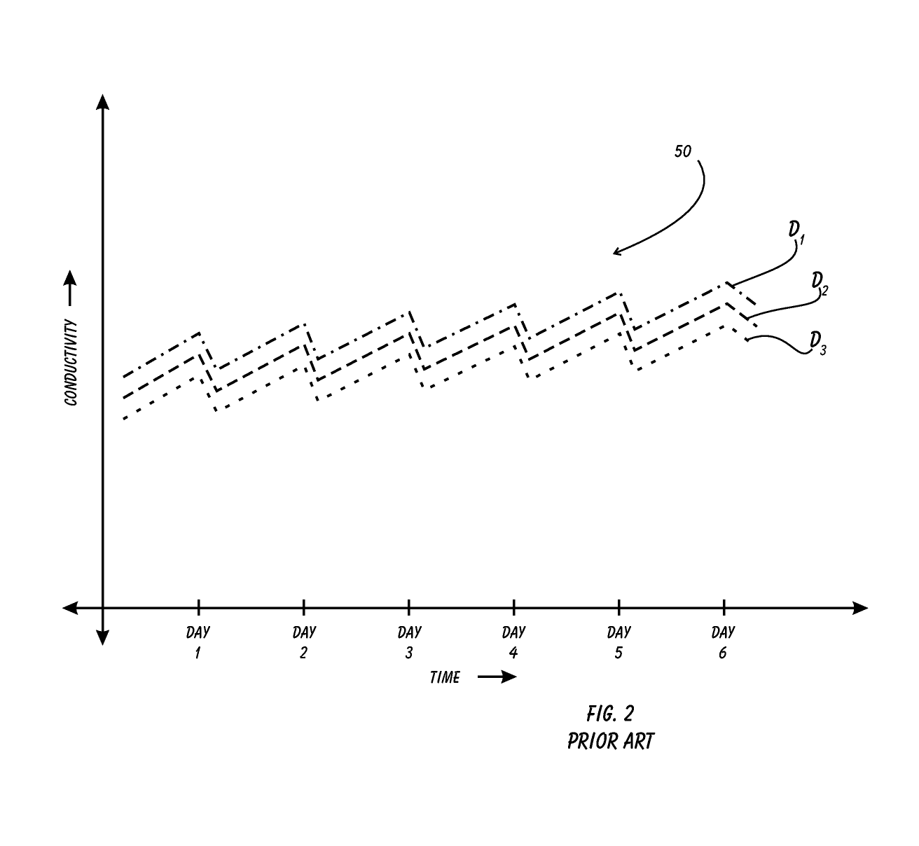

FIG. 3 is an example of an irrigation template for corn;

FIG. 4 is a detailed view of an advanced version of the soil moisture curve;

FIG. 5 is a second view of an irrigation peak of the curve of FIG. 4;

FIG. 6 is a series of irrigation curves as in FIG. 5;

FIG. 7 is a flowchart depicting a preferred embodiment of the method for determining soil refill point of the present invention; and

FIG. 8 is a preferred embodiment of the method for determining crop root depth of the present invention.

### DETAILED DESCRIPTION OF THE PREFERRED EMBODIMENTS

The following description is provided to enable any person skilled in the art to make and use the invention and sets forth the best modes contemplated by the inventor(s) of carrying out his invention. Various modifications, however, will remain readily apparent to those skilled in the art, since the generic principles of the present invention have been defined herein specifically to provide a Method and System for Automated Data Analysis of Soil Moisture.

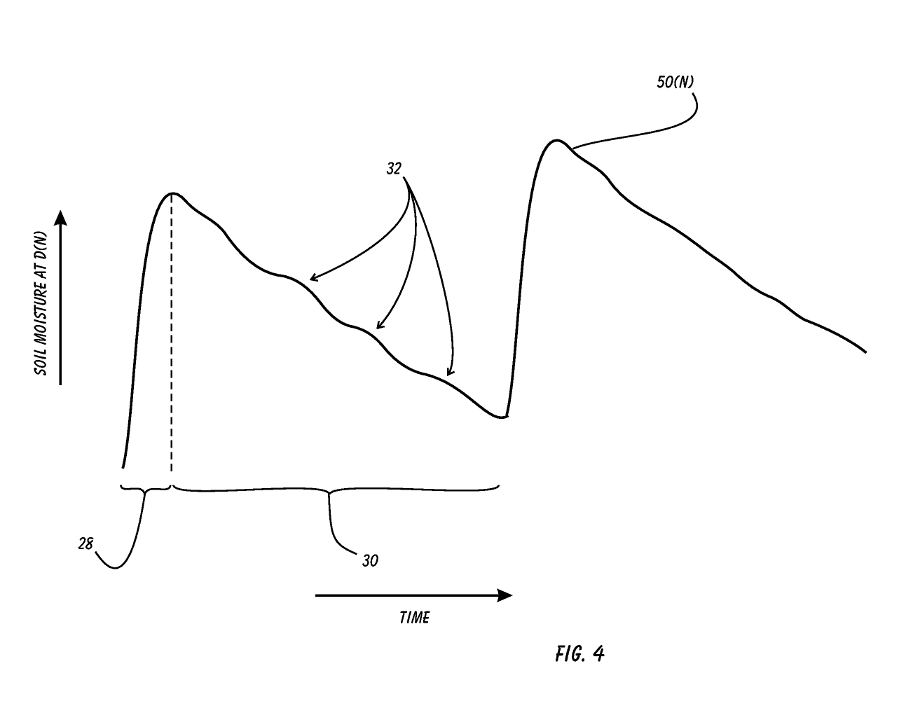

The present invention can best be understood by initial consideration of FIG. 4. FIG. 4 is a detailed view of an advanced version of the soil moisture curve for a particular moisture sensor [12] at depth “n.” It should be recognized that the accuracy, consistency, and general reliability of the example trace shown here is the product of an advanced soil moisture probe developed by Applicant. Other prior probes may not be able to provide the same level of detail, and therefore the analysis discussed herein below is unlikely to be available either.

This example trace shows the soil moisture trend for a pair of sequential irrigation cycles. In the typical corporate farm environment, the fields are irrigated every three days, so it is typical to see a curve that exhibits the features shown here.

On the left, we see the rapid upward trend of the moisture during the irrigation period 28. Once irrigation is ceased, the moisture level in the soil will gradually become lower and lower as the plants consume the water. Evaporation is a factor in the moisture reduction as well, but less-so as the soil depth increases.

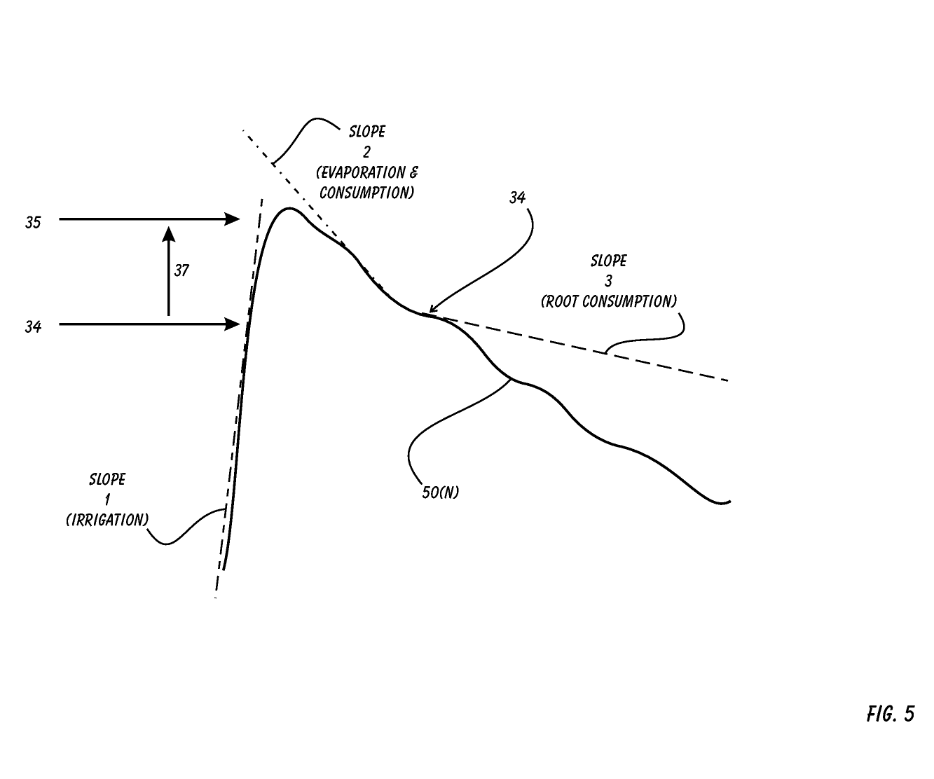

During the consumption period 30, we expect to see a series of “stair-steps” or perturbations 32 in the curve. These perturbations 32 reflect changes in the overall slope of the moisture curve 50(n) as the moisture level is decreasing. While some of this performance could be considered noise, the important process that creates these “steps” is the change in consumption rate of the plants due to the cycle between day and night. During the daytime, the plants will grow, and the roots will consume water from the surrounding soil. During the nighttime, plant growth slows or stops, and therefore water consumption from the soil slows or stops. Therefore, if the roots of the plants in the field being monitored have reached the depth of sensor (n), then the moisture curve should show one perturbation per day. On the contrary, if the roots have not yet reached the depth of sensor (n), then there won't be a full set (or any) of the perturbations 32. FIG. 5 breaks down this analysis further.

FIG. 5 is a second view of an irrigation peak of the curve of FIG. 4. For any soil moisture sensor moisture curve 50(n)². During irrigation, a the curve follows a steep upward direction—Slope 1. Upon cessation of irrigation, the evaporation/consumption begins and a the curve 50(n) follows a sharp downward slope—Slope 2. The first “stair step” or perturbation [32] indicates a reduction in the rate at which moisture is leaving the soil—Slope 3 (Slope 3 has a flatter slope than Slope 2). This slope change indicates that roots are present in the vicinity of the particular moisture sensor (i.e. at its depth), because the plants have entered the nighttime stage of slower growth and slower water consumption from the soil. Presumably, this means that the reduction in moisture level is mostly due to evaporation, rather than from root consumption. ²(n) refers to soil mosture sensor (n) at a pre-determined depth for that sensor.

The moisture level in the soil at the point (and depth) where the roots are present and consuming moisture is known as the “full point” 34 of the soil, because the soil is considered to be “full” with moisture at that depth to support root growth. If the soil moisture level is higher than the full point 34, then the peak moisture 35 will be above the full point 34. This difference signifies over-irrigation 37. Conversely, if the peak moisture level 35 is below the full point 34, then the roots will not be receiving the irrigation that they require (under-irrigation condition). The “holy grail” of crop soil moisture control is the ability to identify the full point 34 of the soil in real time, through all plant development stages and all root (and soil) depths.

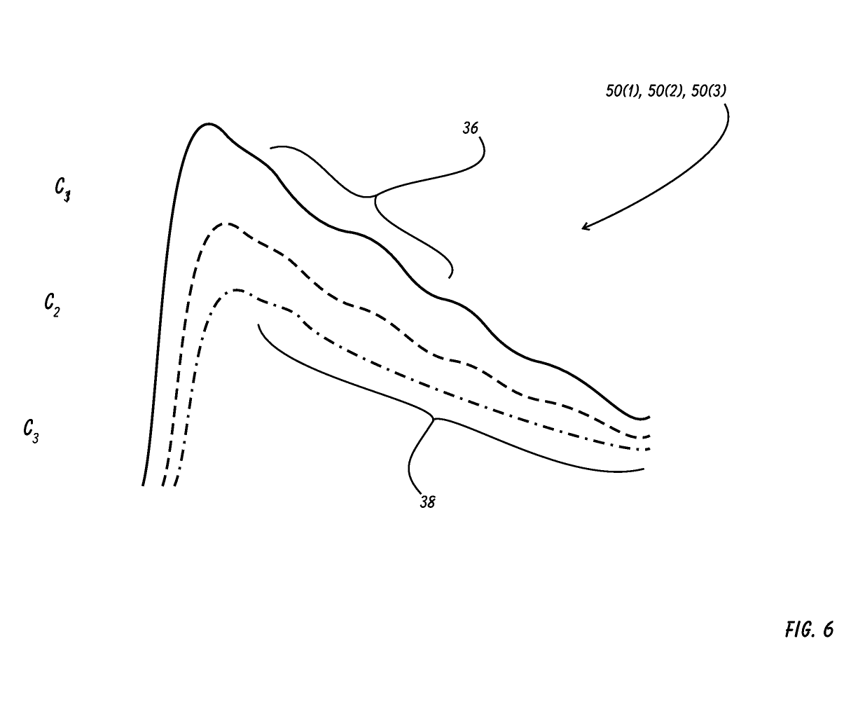

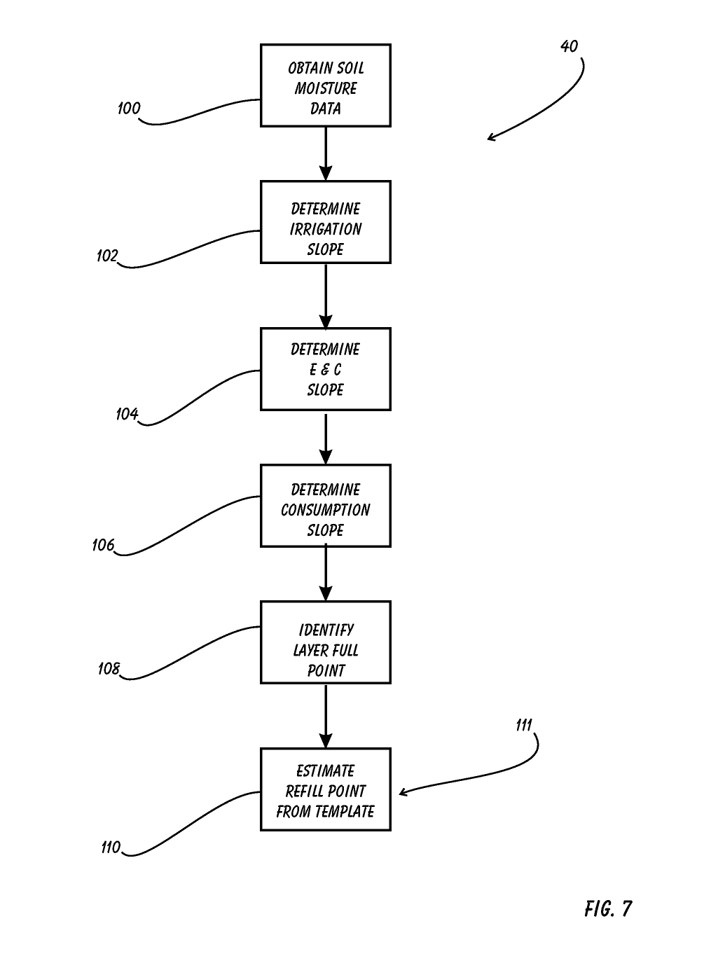

As shown in FIG. 6, where three soil moisture curves 50(1), 50(2) and 50(3) are represented, there are perturbations in stair-step region 36 in the curve 50(1) from the shallowest moisture sensor [12], but there are no perturbations [32] in curve 50(3), which is the soil moisture as measured by a deeper sensor [12]. The “smooth region” 38 in the curve 50(3) indicates that there are no roots present at the depth of sensor [12] 50(3). If we now turn to FIG. 7, we can examine the novel steps of the method of the present invention.

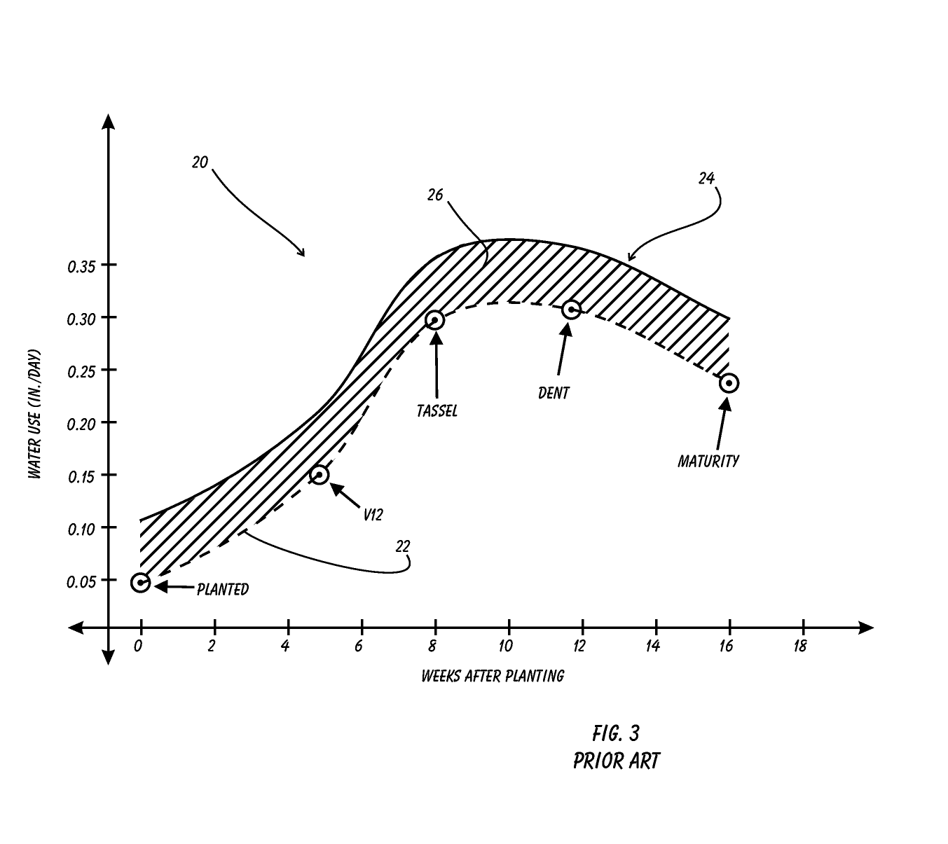

FIG. 7 is a flowchart depicting a preferred embodiment of the method for determining soil full point of the present invention 40. This point can also be referred to as the “refill point,” since it is the point at which the soil should optimally be irrigated to support root consumption. The difference is that the “full point” is identified from measured soil moisture, whereas “refill point” is an estimate of that point to which the soil must be irrigated according to the irrigation template (e.g. see FIG. 3). The method 40 is expected to be ² (n) refers to soil mosture sensor (n) at a pre-determined depth for that sensor. performed by a programmable computer coupled to receive data from one or more soil moisture sensors [12].

First, the soil moisture data is obtained 100. As mentioned previously, this data is assumed to be of the reliability and accuracy of the advanced soil moisture probe of Applicant. Next, the irrigation slope is determined 102. The evaporation/consumption slope is then determined 104 as the slope of the curve after the peak moisture point [35]. The root consumption slope 106 is then determined as the next change in the slope of the moisture curve, where the slope of the curve is reduced. The moisture level where the root consumption is first detected is the full point, and therefore identification of the moisture level of the slope change from evaporation/consumption to consumption is also the identification of the full point 108.

Finally, the refill point is estimated from the template 110, and the optimum irrigation region [26] is moved up or down (i.e. the recommended irrigation quantity is increased or decreased) based on what the actual measured soil moisture and root depth of the plants are. Alternatively, another approach is to estimate the refill point from the full point identified in step 108. This approach might involve setting the refill point at a percentage of the full point (at the different crop stages) 111. The goal would be to identify the irrigation curve (e.g. 22 of FIG. 3), and then estimate the optimum irrigation region [26] as being some percentage above the irrigation curve (the irrigation curve is the full point of the crops as plotted against time for the growth progression of a particular crop).

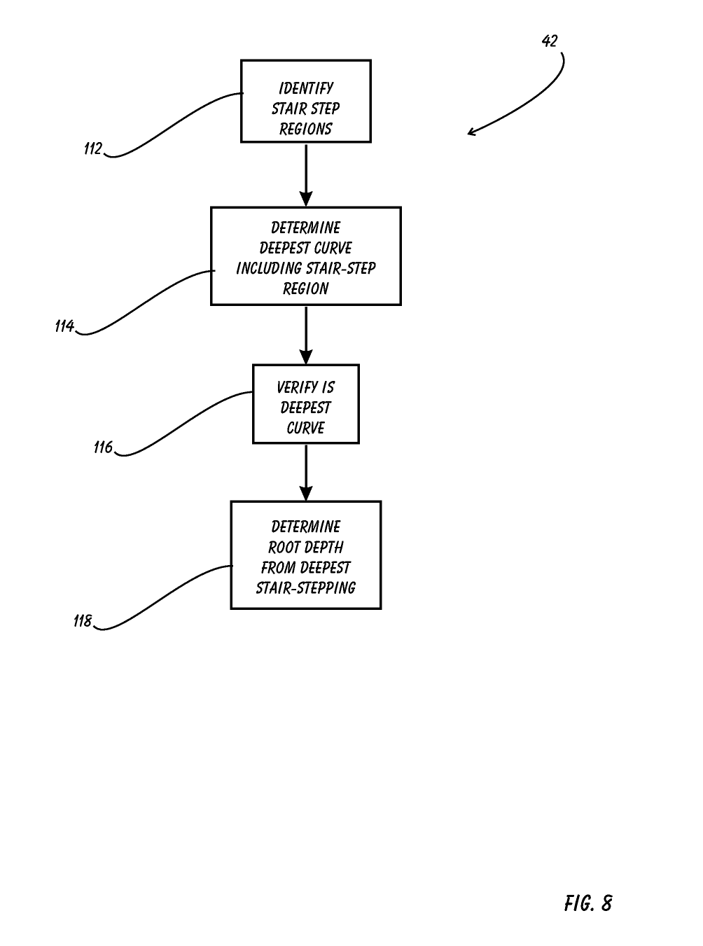

Unless adjusted as suggested herein, the farmer will be guessing whether the soil moisture level at the actual root depth is optimum for the amount of moisture that the plant seeks to consume for the plants' growth stage. The basic ability to determine the depth of the roots is shown in FIG. 8.

FIG. 8 is a preferred embodiment of the method for determining crop root depth 42 of the present invention. First, the stair-step regions [36] are identified in all soil moisture curves [50(n)] 112. Within these results, the curve for the deepest moisture sensor [12] that includes the stair-step region [36] is identified 114. In order to avoid a “false” result that might indicate that the root depth is shallower than it really is, curves [50(n)] for the next three (or other number) of deeper curves [50(n)] are scrutinized to confirm that there are no stair-step regions [36] in any of the curves for these deeper sensors [12] 116. If stair-step regions [36] are found in curves for deeper sensors [12], then the verfication step 16 is repeated (i.e. curves from even deeper sensors are analyzed).

Once verified 116, the actual crop root depth 118 is identified responsive to steps 114 and 116. As discussed previously, this information is very valuable in arriving at the optimum irrigation quantity for the crops as the plants proceed through their growth phases.

Those skilled in the art will appreciate that various adaptations and modifications of the just-described preferred embodiment can be configured without departing from the scope and spirit of the invention. Therefore, it is to be understood that, within the scope of the appended claims, the invention may be practiced other than as specifically described herein.

What is claimed is:

1. A method for determining the depth of roots of plants growing in a zone of soil, comprising the steps of:
irrigating said soil zone;
monitoring the moisture content of the soil in said zone at predetermined depths;
determining a first moisture change rate at two or more said predetermined depths;
determining a second moisture change rate at said two or more predetermined depths; and
identifying the depth of said plant roots in said zone as the deepest said predetermined depth being monitored wherein both said first and second moisture change rates is evident.

2. A method for irrigating a zone of a planted field, comprising the steps of:
irrigating the soil in said zone;
monitoring the soil moisture content at predetermined depths within said zone;
determining a refill point of said soil in said zone responsive to said monitoring; and
second irrigating said soil responsive to said refill point determining.

3. The method of claim 2, wherein said refill point determining comprises the steps of:
determining a peak moisture point of said soil at said predetermined depths;
identifying a moisture rate change perturbation of said soil moisture at said predetermined depths;
identifying the deepest said predetermined depth at which no said perturbation can be identified; and
identifying said refill point responsive to said identified deepest predetermined depth at which no said perturbation can be identified.

4. The method of claim 3, wherein said refill point identifying comprises:
obtaining an irrigation template for said plant and said zone; and
estimating said refill point responsive to said depth at which no said perturbation can be identified and said irrigation template.

5. The method of claim 3, wherein said refill point identifying comprises:
identifying a full point responsive to said depth at which no said perturbation can be identified; and
estimating said refill point as being equal to a percentage of said full point.

6. A method for optimizing the irrigation of a zone of a planted field, comprising the steps of:
first irrigating the soil and plants in said zone;
monitoring the soil moisture content at a plurality of predetermined depths within said zone;
determining a peak moisture point of said soil at said plurality of predetermined depths;
identifying a moisture rate change perturbation of said soil moisture at said predetermined depths, said perturbations identifed by:
determining a first moisture change rate after said peak moisture point at said predetermined depths; and
determining a second moisture change rate after said first moisture change rate at said predetermined depths;
identifying the deepest said predetermined depth at which no said perturbation can be identified; and
second irrigating responsive to said identifying of said deepest said predetermined depth at which no said perturbation can be identified.

7. The method of claim 6, wherein:
said second irrigating is responsive to identifying a refill point.

8. The method of claim 7, wherein said refill point identifying comprises:
obtaining an irrigation template for said plant and said zone; and
estimating said refill point responsive to said depth at which no said perturbation can be identified and said irrigation template.

9. The method of claim 7, wherein said refill point identifying comprises:
identifying a full point responsive to said depth at which no said perturbation can be identified; and
estimating said refill point as being equal to a percentage of said full point.
